(19) United States
(12) Patent Application Publication
(10) Pub. No.: US 2026/0363592 A1
(43) Pub. Date: Nov. 19, 2026

(54) SYSTEM AND METHOD FOR ASSESSMENT AND/OR REHABILITATION OF PATIENTS

(71) Applicant: RAMBAM MED-TECH LTD., Haifa (IL)

(72) Inventors: Goded SHAHAF, Haifa (IL); Dana BARON SHAHAF, Haifa (IL)

(73) Assignee: RAMBAM MED-TECH LTD., Haifa (IL)

(21) Appl. No.: 19/132,088

(22) PCT Filed: Nov. 23, 2023
(86) PCT No.: PCT/IL2023/051211

§ 371 Date: May 22, 2025

Related U.S. Application Data
(60) Provisional application No. 63/427,908, filed on Nov. 25, 2022.

(51) Int. Cl. A61B 5/16; A61B 5/38; A61B 5/389; A61B 5/398

(52) U.S. Cl. CPC A61B 5/168; A61B 5/163; A61B 5/38; A61B 5/389; A61B 5/398; A61B 2505/09

(57) ABSTRACT

Aspects of the invention directed to a method of a Disorder of Consciousness (DOC) rehabilitation are disclosed. The method comprises (a) providing a patient, a first audio stimulation during a first predetermined duration; (b) at the end of the first audio stimulation, providing the patient instruction to move facial or eye muscles in order to replay the audio stimulation; and (c) replaying the first audio stimulation if a signal indicative of a voluntary response of the facial or eye muscles is received.

### CROSS REFERENCE TO RELATED APPLICATIONS

This application is a PCT Patent Application which claims the benefit of priority under 35 U.S.C. § 119(e) of U.S. Provisional Patent Application No. 63/427,908, filed 25 Nov. 2022. The contents of the above applications are all incorporated by reference as if fully set forth herein in their entirety.

### FIELD OF THE INVENTION

The present invention relates generally to methods of assessment and/or rehabilitation. More specifically, the present invention relates to a system and method for assessment and rehabilitation of patients with a consciousness disorder, chronic pain, stress or anxiety, and/or cognitive decline.

### BACKGROUND OF THE INVENTION

Rehabilitation depends on patient's engagement. Two barriers are generally described as reducing the patient's engagement during rehabilitation-reduced cognitive effort, and enhanced stress effect. It is important to monitor these barriers for any type of rehabilitation. However, it is especially crucial to monitor these barriers in specific cases, in which reduced cognitive effort or enhanced stress effect are the major factors, which hinder the success of rehabilitation, and/or are difficult to assess by clinical observation, and/or a simple treatment might overcome these barriers and advance the patient's rehabilitation dramatically.

Measuring electrophysiological or physiological signals of patients using, for example, an Electroencephalogram (EEG) system, an Electromyography (EMG) system, electrooculogram (EOG); or an eye-tracking system are known methods for diagnosing cognitive, mental, or any other brain related dysfunctions.

There is little to no use of these methods as feedback in rehabilitation treatments for identifying the barriers of reduced cognitive effort, or of enhanced stress effect.

EEG, EMG, EOG and eye-tracking systems are relatively simple devices that can be incorporated into a simple system that can be operated by the patient itself, or by non-professional caregivers, in the clinic or at home.

Accordingly, there is a need for a method of using EEG, EMG, EOG and eye-tracking systems as feedback regarding cognitive effort and stress effect in automatic diagnosis and in automatic computerized rehabilitation process.

### SUMMARY OF THE INVENTION

Some aspects of the invention may be directed to a method of a Disorder of Consciousness (DOC) rehabilitation comprising:
a. providing a patient, a first audio stimulation during a first predetermined duration;
b. at the end of the first audio stimulation, providing the patient instruction to move facial or eye muscles in order to replay the audio stimulation;
c. replaying the first audio stimulation if a signal indicative of a voluntary response of the facial or eye muscles is received.

In some embodiments, the method may further include:
a. continuously receiving electrophysiological or physiological signals of a patient from an electrophysiological measuring system or a physiological measuring system selected from, an Electroencephalogram (EEG) system, an Electromyography (EMG) system, electrooculogram (EOG); or an eye-tracking system,
b. continuously extracting attention indexes from the electrophysiological or physiological signals;
c. detecting a first attention index higher than a threshold value;
d. if the signal indicative of the voluntary responses was not received, providing the patient additional instruction to move facial or eye muscles in order to replay the audio stimulation;
e. replaying the first audio stimulation if a signal indicative of a voluntary response of the facial or eye muscles is received.

In some embodiments, the method may further include:
a. continuously receiving electrophysiological or physiological signals of a patient from an electrophysiological measuring system or a physiological measuring system selected from, an Electroencephalogram (EEG) system, an Electromyography (EMG) system, electrooculogram (EOG); or an eye-tracking system,
b. continuously extracting attention indexes from the electrophysiological or physiological signals;
c. detecting a second attention index lower than the threshold value;
d. if the signal indicative of the voluntary responses was not received, providing to the patient an evoking stimulation configured to evoke a response of the facial or eye muscles;
e. replaying the first audio stimulation.

In some embodiments, the signal indicative of the voluntary response is received from the electrophysiological or physiological measuring system. In some embodiments, the signal indicative of the voluntary response is received from the same channel as the electrophysiological or physiological signals. In some embodiments, the instruction to move the facial or eye muscles is provided as one of: audio instruction, visual instruction, and muscle stimulation. In some embodiments, the muscle stimulation is provided by an electrophysiological measuring system or a physiological measuring system. In some embodiments, the voluntary responses of the facial or eye muscles comprising at least one of: blinking, closing the eyes, raising the eyebrows, directing gaze, smiling, opening and closing the mouth.

In some embodiments, the method may further include:
a. providing the patient a second audio stimulation during a second predetermined duration;
b. during the second audio stimulation, providing the patient instruction to relax the facial muscle in order to stop the second audio stimulation;
c. stop playing the second audio stimulation if a signal indicative of relaxing the facial muscle is received from the electrophysiological or physiological measuring system.

In some embodiments, the second audio stimulation includes an unpleasant sound. In some embodiments, the instruction to relax the facial muscle is provided as one of: audio instruction, visual instruction, and muscle stimulation. In some embodiments, the method may further include providing to user instructions to associate the moving of facial or eye muscles with the word YES, and relaxing the facial muscle with the word NO.

In some embodiments, the extraction of an attention index comprises dividing the electrophysiological signal into a plurality of segments and dividing each of the segments into a plurality of epochs. In some embodiments, the duration of each of the plurality of the segments is in the range of 2.5 to 10 seconds and the duration of each of the epochs is in the range 0.2 to 1 seconds.

Some additional aspects of the invention may be related to a method of rehabilitation of at least one of chronic pain, stress, and anxiety, comprising:
a. continuously receiving electrophysiological or physiological signals of a patient from an electrophysiological measuring system or a physiological measuring system selected from, an Electroencephalogram (EEG) system, an Electromyography (EMG) system, electrooculogram (EOG); or an eye-tracking system,
b. continuously extracting alertness indexes from the electrophysiological signals;
c. providing to the patient instructions to perform a first task;
d. determining if the extracted alertness indexes is at an effective alertness range during the performance of the first task; and
e. providing the patient instructions to perform a second task, more advanced than the first task.

In some embodiments, the method may further include:
a. continuously extracting attention indexes from the electrophysiological or physiological signals;
b. identifying in patterns in the attention indexes and the alertness indexes related to one of the following barriers:
   I. discomfort barrier when the attention indexes is below an attention threshold values and the alertness indexes is above the effective alertness range;
   II. dissociation barrier when the attention indexes is below a threshold value and the alertness indexes is below the effective alertness range;
   III. coping under pain barrier when the alertness indexes is above the effective alertness range and the attention indexes is above the threshold value; and
   IV. dominant avoidance barrier when the alertness indexes is at the effective alertness range and the attention indexes is one of: below the threshold value, and above the threshold value while having momentary decreases in the attention indexes to below the threshold value.

In some embodiments, the method may further include providing instructions to the patients based on the barrier. In some embodiments, the instructions are selected from:
i. actively relaxing while performing the first task;
ii. rest for a first predetermined amount of time;
iii. perform a third task, less advanced than the first task;
iv. rest for a second predetermined amount of time and repeat the first task.

In some embodiments, the selection, duration and repetition of the provided instructions are determined based on the barrier. In some embodiments, the alertness index is related to at least one of, pain, anxiety, and stress.

Some additional aspects of the invention may be directed to a method of post-operative rehabilitation, comprising:
a. continuously receiving electrophysiological or physiological signals of a patient from an electrophysiological measuring system or a physiological measuring system selected from, an Electroencephalogram (EEG) system, an Electromyography (EMG) system, electrooculogram (EOG); or an eye-tracking system,
b. continuously extracting attention indexes from the electrophysiological or physiological signals;
c. providing to the patient instructions to perform a first cognitive task;
d. determining a cognitive score for the first cognitive task;
e. determining a first attention level during the first cognitive task, based on the attention index;
f. if the cognitive score is below a required value detecting an emotional barrier when rapid temporal changes are demonstrated in the attention indexes.

In some embodiments, the method may further include:
a. continuously extracting alertness indexes from the electrophysiological or physiological signals;
b. determining a first alertness level during the first cognitive task, based on the alertness index, and wherein if the cognitive score is below a required value, determining if the alertness index is outside an effective alertness range.

In some embodiments, the method may further include: providing to the patient instructions to:
a. relax and breath for a predetermined amount of time if the alertness index is outside the effective alertness range; and
b. repeat the first cognitive task.

In some embodiments, the method may further include:
providing the patient stimulation if the attention index is below an attention threshold level; and
providing the patient instructions to repeat the first cognitive task.

In some embodiments, providing the patient instructions to perform a first cognitive task comprises performing the task on a computer and wherein determining the cognitive score is done by the computer.

In some embodiments, the method may further include: providing to the patient instructions to perform a second cognitive task if the determined cognitive score is above the required value.

Some additional aspects of the invention may be related to a method of pre-operative prediction of POCD, comprising:
a. continuously receiving electrophysiological or physiological signals of a patient from an electrophysiological measuring system or a physiological measuring system selected from, an Electroencephalogram (EEG) system, an Electromyography (EMG) system, electrooculogram (EOG); or an eye-tracking system,
b. continuously extracting attention indexes from the electrophysiological or physiological signals;
c. providing to the patient instructions to perform a pre-operative cognitive task;
d. determining a cognitive score for the pre-operative cognitive task;
e. determining an attention level during the pre-operative cognitive task, based on the attention index;
f. determining a high risk for POCD if the cognitive score is in an intermediate range and the attention level is above an attention threshold value.

In some embodiments, the method may further include: determining the high risk for POCD if no mental barrier was detected in the attention indexes, wherein the mental barrier is defined by rapid temporal changes in the attention indexes.

In some embodiments, the high risk for POCD is further based on at least one of: general cognitive level, pre-operative medical condition and/or demographic data. In some embodiments, providing the patient instructions to perform the cognitive task comprises performing the task on a computer and wherein determining the cognitive score is done by the computer.

Some additional aspects of the invention are directed to: a method for evaluating a validity of a cognitive test, comprising:
a. continuously receiving electrophysiological or physiological signals of a patient from an electrophysiological measuring system or a physiological measuring system selected from, an Electroencephalogram (EEG) system, an Electromyography (EMG) system, electrooculogram (EOG); or an eye-tracking system,
b. continuously extracting attention indexes from the electrophysiological or physiological signals;
c. continuously extracting alertness indexes from the electrophysiological or physiological signals;
d. providing to the patient instructions to perform a cognitive task;
e. determining a cognitive score for the first cognitive task;
f. determining an attention level during the first cognitive task, based on the attention index;
g. determining an alertness level during the first cognitive task, based on the alertness index; and
h. determining that the score is valid if the attention level is within an effective attention range and the alertness level is within an effective alertness range.

In some embodiments, the method may further include: determining that the score is valid if no emotional barrier was detected in the attention indexes, wherein the emotional barrier is defined by rapid temporal changes in the attention indexes. In some embodiments, providing the patient instructions to perform the cognitive task comprises performing the task on a computer and wherein determining the cognitive score is done by the computer.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a block diagram of a system for assessment and/or rehabilitation of a patient according to some embodiments of the invention;

FIG. 1B is a block diagram, depicting a computing device which may be included in a system for assessment and/or rehabilitation of a patient according to some embodiments of the invention;

FIG. 2 is a flowchart of a method of a Disorder of Consciousness (DOC) rehabilitation according to some embodiments of the invention;

FIG. 3A shows a nonlimiting example for electrophysiological signals and the extraction of attention indexes according to some embodiments of the invention;

FIG. 3B is graph of a nonlimiting example of attention indexes being indicative of reduced consciousness in patients according to some embodiments of the invention;

FIGS. 4A and 4B are graphs showing the effect of music and the learning curve on the attention indexes of patients with reduced consciousness according to some embodiments of the invention;

FIGS. 5A and 5B are graphs showing the effect of teaching the patient an “avoid” or NO command on the attention indexes of patients with reduced consciousness according to some embodiments of the invention;

FIG. 6A is a flowchart of a method of rehabilitation of at least one of chronic pain, stress, and anxiety according to some embodiments of the invention;

FIG. 6B shows nonlimiting examples for EEG data taken during tension and relaxed period according to some embodiments of the invention.

FIG. 7 shows graphs showing the alertness index (top graph) and the attention index (bottom graph) of a patient at rest according to some embodiments of the invention;

FIG. 8A shows graphs showing the alertness index (top graph) and the attention index (bottom graph) of a patient during a discomfort/pain barrier according to some embodiments of the invention;

FIG. 8B shows graphs showing the alertness index (top graph) and the attention index (bottom graph) of a patient during a dissociation barrier according to some embodiments of the invention;

FIG. 8C shows graphs showing the alertness index (top graph) and the attention index (bottom graph) of a patient during coping under discomfort/pain barrier according to some embodiments of the invention;

FIG. 8D shows graphs showing the alertness index (top graph) and the attention index (bottom graph) of a patient during a dominant avoidance barrier according to some embodiments of the invention;

FIG. 9 is a flowchart of a method of post-operative cognitive diagnosis for rehabilitation according to some embodiments of the invention;

FIG. 10A is a graph showing the attention indexes of post-surgery patients according to some embodiments of the invention;

FIGS. 10B and 10C include graphs showing post-operative cognitive rehabilitation results according to some embodiments of the invention;

FIG. 10D includes images of a system for post-surgery cognitive tests and rehabilitation according to some embodiments of the invention;

FIG. 11A is a flowchart of a method of pre-operative prediction of post-operative cognitive dysfunction or decline (POCD) according to some embodiments of the invention;

FIG. 11B shows the results of pre-operative prediction of POCD) according to some embodiments of the invention;

FIG. 12 is a flowchart of evaluating a validity of a cognitive test according to some embodiments of the invention; and

FIGS. 13A, 13B and 13C are graphs showing several cognitive scores and attention taken during different levels of attention and alertness according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Some embodiments of the present invention disclose a method and a system for automatic computer-based applications/treatments for assessment and/or rehabilitation of patients with a consciousness disorder. Some additional embodiments may be related to the rehabilitation of chronic pain, stress, or anxiety. Some additional embodiments may be related to the rehabilitation of POCD. Some additional embodiments may be related to pre-operative prediction of POCD and to evaluating a validity of a cognitive test taken pre or poste operation.

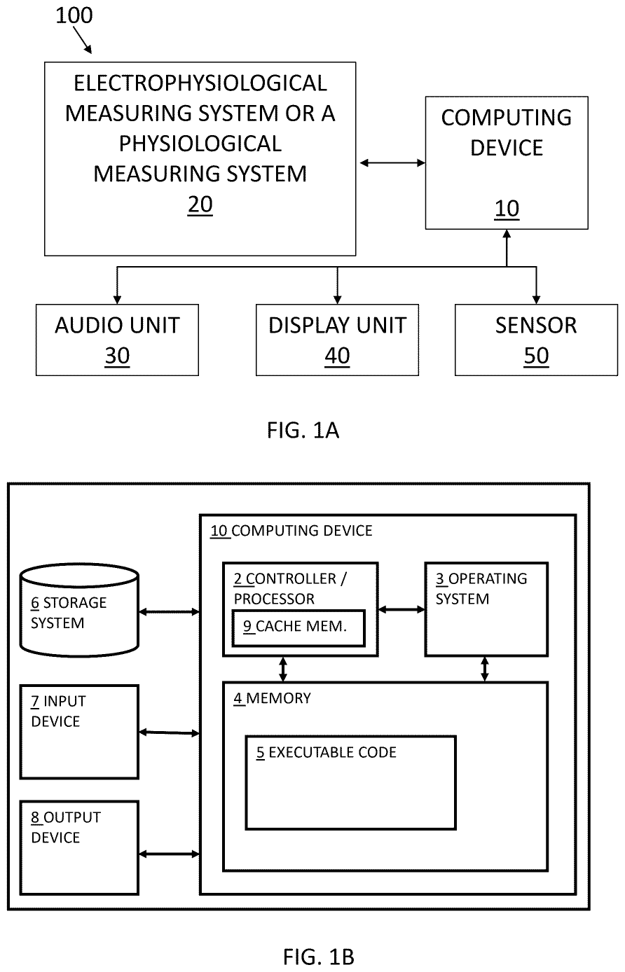

Reference is now made to FIG. 1A which is a block diagram of a system for assessment and/or rehabilitation of a patient according to some embodiments of the invention. A system 100 may be used for DOC rehabilitation, for rehabilitation of at least one of chronic pain, stress, and anxiety, and for post-operative rehabilitation. In some embodiments, system 100 may be used for pre-operative prediction of POCD and/or for evaluating a validity of a cognitive test.

System 100 may include a computing device 10 discussed in detail with respect to FIG. 1B. In some embodiments, system 100 may include an electrophysiological measuring system or a physiological measuring system 20 for measuring electrophysiological or physiological signals of a patient. Measuring system 20 may be selected from an Electroencephalogram (EEG) system, an Electromyography (EMG) system, electrooculogram (EOG), an eye-tracking system and the like.

In some embodiments, system 100 may further include an audio unit 30 configured to play audio stimulation and/or audio instructions. Audio unit 30 may include one or more loudspeakers and/or one or more headphones. Audio unit 30 may be in communication with computing device 10 or may be included in output device 8 of computing device 10.

In some embodiments, system 100 may include at least one display unit 40. Display unit 40 may include any type of screen (e.g., a LED screen, touchscreen, etc.) that may be in communication with computing device 10 or may be included in output device 8 of computing device 10.

In some embodiment, system 100 may further include one or more sensors 50. Sensor 50 may be any sensor configured to measure physiological parameters of the patients, for example, heartrate monitor, blood saturation level, blood pressure monitor, breathing sensors (e.g., breathing biofeedback sensors), sweat gland activity sensors, temperature sensors and the like.

Reference is now made to FIG. 1B, which is a block diagram depicting a computing device, which may be included within an embodiment of a system for assessment and/or rehabilitation of a patient, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

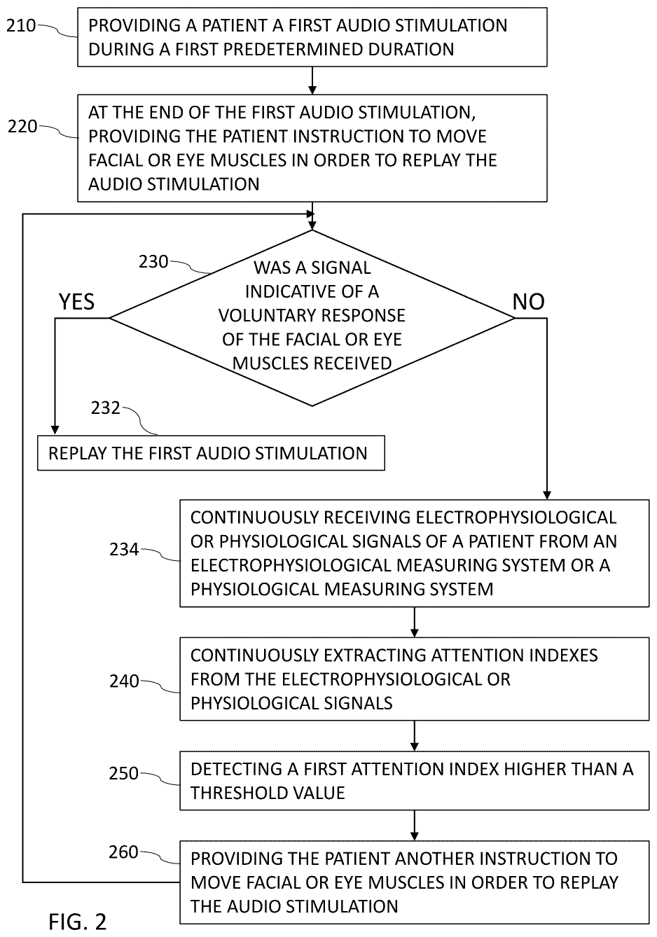

Executable code 5 may be any executable code, e.g., an application, a program, a process, task or script. Executable code 5 may be executed by processor or controller 2 possibly under control of operating system 3. For example, executable code 5 may be an application as discussed with respect to FIGS. 2, 6, 9, 11 and 12 as further described herein. Although, for the sake of clarity, a single item of executable code 5 is shown in FIG. 1B, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Medical records and historical alertness and attention indexes (e.g., tension index, cognitive effort, etc.) may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

Reference is now made to FIG. 2, which is a flowchart of a method for rehabilitation of Disorder of Consciousness (DOC) according to some embodiments of the invention. The method of FIG. 2 may be executed by computing device 10 of system 100.

In step 210 the method may include providing to a patient, a first audio stimulation during a first predetermined duration. In some embodiments, the patient may be a patient diagnosed with a DOC. For example, computing device 10 (e.g., controller 2) may control audio unit 30 to play the first audio stimulation for 10-30 seconds. The first audio stimulation may be selected form, a tune the patient likes, a voice of a loved one, and the like. In some embodiments, the duration may be determined based on the type of stimulation and the patient.

In step 220, at the end of the first audio stimulation, the method may include providing the patient instruction to move facial or eye muscles in order to replay the audio stimulation. In some embodiments, the instructions may include a request to do at least one of: blink, open/close the mouth, smile, close the eyes, raise eyebrows, directing gazing and the like. For example, controller 2 may control audio unit 30 to play verbal/audio instructions asking the patient to blink in order to replay the audio stimulation. In yet another example, controller 2 may control display unit 40 to display a text asking the patient to move facial or eye muscles (e.g., blinking), show the patient a video demonstrating blinking/smiling, showing the patient an image of a smiling/blinking man and the like. In yet another example, controller 2 may control electrophysiological measuring system or physiological measuring system 20 to provide a muscle stimulation, e.g., using the EEG or EMG electrodes to provide electrical stimulation that may stimulate the face muscle to blink/raise eyebrows and the like.

In step 230, if a signal indicative of a voluntary response of the facial or eye muscles is received (Step 230—YES) the method may include replaying the first audio stimulation (Step 232).

In some embodiments, if the signal indicative of the voluntary responses was not received (Step 230—NO), the method may include Step 234 of continuously receiving electrophysiological or physiological signals of a patient from an electrophysiological measuring system or a physiological measuring system. For example, the patient may be wearing EEG, EMG, EOG electrodes or the eyes of the patient may be tracked by an eye tracker. Therefore, controller 2 may continuously receive electrophysiological or physiological signals of the patient.

In some embodiments, these measurements may be conducted following or in parallel to steps 210 to 230. In some embodiments, the measurements may be conducted using a single channel, for example, a single EEG, EMG, or EOG channel, or a single eye tracker. In some embodiments, the same EEG, EMG, or EOG channel may be used for providing the electrical stimulation in step 230. Therefore, system 100 may require using only one channel of system 20.

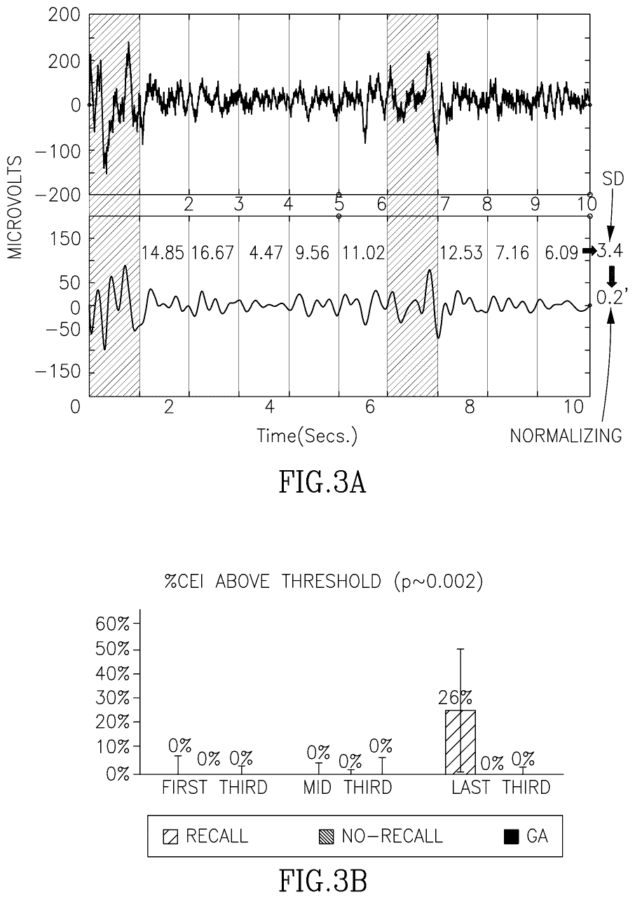

In step 240, the method may include continuously extracting attention indexes from the electrophysiological or physiological signals. A nonlimiting example, for extracting an attention index, such as, a cognitive effort index (CEI) from an EEG signal is given in FIG. 3A. Other optional attention indexes known in the art may include the following markers:

Power and power ratio based markers: It was found that greater attention often involves greater activity power in higher EEG frequency bands (e.g. beta—~13 Hz-~30 Hz) and reduced attention often involves greater activity power in lower frequency bands (e.g. theta—~4 Hz-~7 Hz). Activity power could be computed in multiple ways—e.g. Fourier-based power analysis, integral of absolute values, etc. Furthermore, it is customary to compute the ratio between a lower frequency (e.g. theta) and the higher frequency (e.g. beta) as an indication of lower attention (and vice versa).

Synchronization based markers: It was found that greater attention often involves synchronization between distant electrodes for some EEG frequency bands (e.g. theta—~4 Hz-~7 Hz, beta—~13 Hz-~30 Hz and gamma—>~30 Hz) and desynchronization for other frequency bands (e.g. alpha—~8 Hz-~12 Hz). The synchronization/desynchronization could be computed between any two electrophysiological channels on the head—whether ipsilateral (on the same side) or contralateral.

Synchronization can be computed in various methods. It may be based on any correlation index between the simultaneous sampling points of the contralateral channels—for example Pearson correlation or coherence analysis. Furthermore, it is possible to transform the raw signals before the synchrony analysis—for example to look at synchronization in specific frequency bands—e.g. delta, theta, alpha, beta or gamma—or their combination. It is also possible to analyze synchronization between transformations of the raw signals by template matching, wavelet analysis and components analysis. Synchronization can also be computed on the basis of the ratio between the level of activity on both sides, or their power in any frequency band, or combination of frequency bands.

It is also possible to transform the signal into discrete values and to evaluate correlation between these discrete values. For one example, it is possible to transform the signal to positive and negative deflections from the average during the selected timeframe and compute the hamming distance as an index for correlation. But it is also possible to divide the signal into more discrete values.

Template-matching based markers: Multiple pattern templates were identified as related to attention. These templates could involve any combination of frequency bands and wave patterns. They could be derived from data mining methods, which identify them as related with an experimental condition, which is expected to involve attention. See for example: Monitoring attention in ADHD with an easy-to-use electrophysiological index. At times, they are derived from association to previously identified markers in EEG or in other modalities.

These markers are then sought in the sampled electrophysiological activity. Their matching with the sampled activity might be based on various methods of signal correlation—such as Fourier-based correlation and computation of average distance between the template and a matched-size moving window in the sampled data. The matching could be done after amplitude normalization of both the template and moving window to an equivalent range. The distance evaluation could be done by computation of the average distance over sampling points, or by any other accepted method of distance evaluation between two sampled signals.

Variability based markers: It has been shown that rapid signal variability between consecutive sample sequences, which last between tens of milliseconds to few seconds, associate with level of attention. For some sequence patterns it seems that reduced inter-sequence variability is associated with a higher level of attention, while for other sequence patterns it seems that increased inter-sequence variability is associated with higher level of attention. This duality stems from a set of factors, such as the frequency band of the activity, which corresponds well with the fact that the activity of some frequency bands increases with attention, while the activity of others decreases. Other factors may include spatial location of the electrophysiological channel etc.

For the analysis of variability, a quantification of each of the consecutive segments could be generated, such as segment's average power, segment's standard deviation, segment's max or min amplitude, segment's power analysis in various frequency band, degree of occurrence of a certain template in the segment, etc. Then variance between the set of segments could be computed—e.g. by variance analysis, standard deviation analysis, inter-percentile distance, etc. Alternatively, it is possible to evaluate the distance of each segment from a template and to compute the average distance among these evaluations, or some other group summary.

Thus, it is possible to compute variability based on a quantification of each segment or based on a distance from a given predetermined value. Either way, the quantifying value of each segment could be normalized to a pre-determined range (e.g. [0,1]) before computing the overall variability.

Blink based markers: It has been shown that blinking is associated with attention. Generally, slower blinking is associated with increased attention. Blinks are recordable from the electrophysiological channels as well as from eye movement sensors. The blinks are identifiable by analysis of large slow frequency (e.g. in the delta range) deflections of more than few tens of microvolts (e.g. more than 30 microvolts) with gaps of few hundred milliseconds (e.g. at-least 500 milliseconds from a previous blink identification). The frequency of the blinks is then calculated as an index for attention. For example, increased attention might be related to a blinking frequency of ~¼ Hz and decreased attention might be related to a blinking frequency of ~½ Hz. Specifically, for blinking, it is also possible to extract them from eye tracker blink detectors.

For all the above types of markers as well as for other electrophysiological markers for attention, prior to the computation of the marker, it is possible to remove from the signal epochs of definite size (e.g. from parts of seconds to a few seconds), which are identified as noisy—e.g. due to large-amplitude waves, or by any other means, or to filter out such epochs—e.g. ECG and only thereafter to compute the index. Then, it is possible to generate a marker only for samples with enough epochs (e.g. more than 50% valid epochs). It is also possible to include in the analysis only specific types of filtered activity—e.g. specific frequency bands, specific wavelets, specific principal or independent components, etc. Finally, it is possible to normalize whichever selected marker to a set range presents one example of an attention marker computation. In this example the computation is variability based, but as stated above, the index can be computed using multiple methods, as detailed in the section above and in the literature.

Reference is now made to FIG. 3A which shows how CEI is extracted from an EEG signal according to some embodiments of the invention. The data may be analyzed, online or offline, in segments of a predefined duration, e.g., 10 seconds, as presented in the top graph. Each segment may be divided to epochs of a predefined duration, for example, 1 second. Noisy epochs may be excluded due to deviant amplitude as marked in the grey-shaded bands. Data may be filtered for a given band, for example, a frequency band (e.g., delta) of 1-4 Hz, as presented in the bottom graph. For the remaining valid epochs, a power index may be computed, for example, in this specific case average absolute amplitude in the delta activity band. The resulting values are presented numerically for each epoch in the bottom graph (e.g., 14.85 for the second epoch, 16.67 for the third epoch, etc.). The standard deviation of these values may be computed as pointed at by the horizontal arrow. This value then may be normalized based on previous samples, as presented by the vertical arrow (division by 12.5 in this example) to generate the attention index.

Referring now to FIG. 3B which is graph of a nonlimiting example of attention indexes being indicative of reduced consciousness in patients according to some embodiments of the invention. The graph shows data collected from 25 patients who underwent deep sedation and 13 patients who underwent general anesthesia. The graph shows that for 11 patients the CEI was above threshold for a noticeable period of the last third of the procedure. These patients recall awareness under sedation. Accordingly, measuring CEI may be indicative even of a very low level of awareness.

In step 250, the method may include detecting a first attention index higher than a threshold value. For example, controller 2 may detect if the normalized attention index is higher than 0.3 (30%) of the maximal attention index has been detected while the audio stimulation was played, meaning that the patient had enough awareness to hear the audio stimulation.

In step 260, the method may include replaying the first audio stimulation if a signal indicative of a voluntary response of the facial or eye muscles is received.

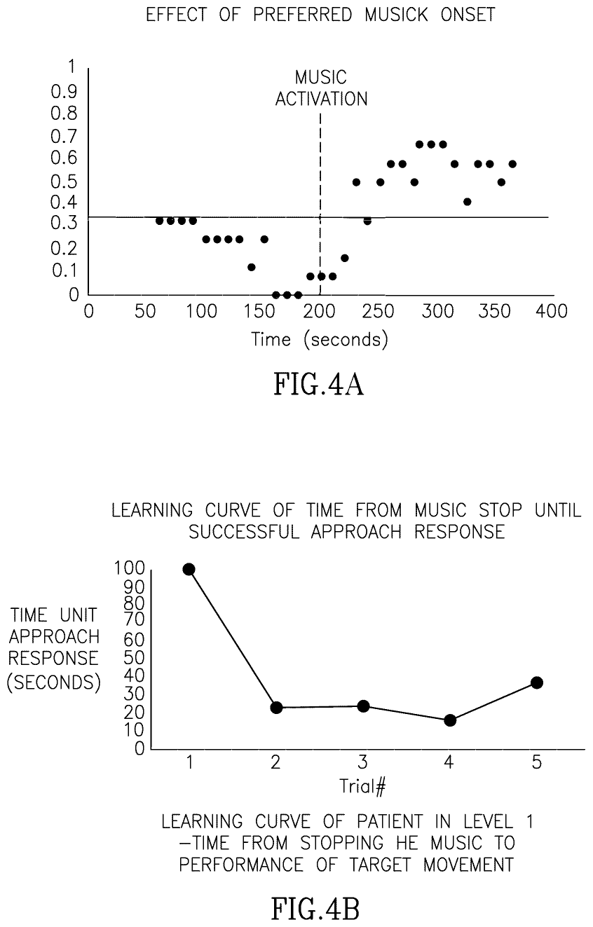

Reference is now made to FIG. 4A which shows the impact of an audio stimulation, such as, musical tune on the attention index of the patient. As clearly shown in the graph, once the musical tune is played there is a rise in the attention index, even if several trails/attempts (e.g., several music intervals) are required until the attention index of the patient exceeds the threshold value. Once the attention index is above the threshold, step 220 may be repeated until a signal indicative of a voluntary response of the facial or eye muscles is received.

Reference is now made to FIG. 4B which shows a learning curve of a patient diagnosed with DOC to activate music using voluntary response of the facial or eye muscles according to some embodiments of the invention. In the nonlimiting example of FIG. 4B, the patient was requested to blink in order to replay the music. As shown in the graph it took the patient 2 trials to understand how to replay the music. In the first trial almost 100 seconds were required for the patient to blink. A much shorter delay between 10 to 30 seconds was observed in trials 2-5.

In some embodiments, steps 230 to 240 may be repeated, and the method may include detecting a second attention index lower than the threshold value. Therefore, in some cases an evoking stimulation configured to evoke a response of the facial or eye muscles may be provided, for example, an electrical stimulation that may activate the facial or eye muscles, or a strong sound (e.g., above 60 dB). The evoking stimulation may be provided by the EEG, EMG or EOG system, using the same or different channel from the one used to receive the indicative signal.

In some embodiments, controller 2 may then replay the first audio stimulation, thereby associating, in the patients' mind, playing the first audio stimulation with an activation of the facial or eye muscles.

In some embodiments, the method may include teaching the patient to stop an audio stimulation. In such cases, the method may include providing the patient a second audio stimulation during a second predetermined duration. For example, the second audio stimulation may include an unpleasant sound. In some embodiments, the method may include during the second audio stimulation, providing the patient instruction to relax the facial muscle in order to stop the second audio stimulation. If a signal indicative of relaxing the facial muscle is received from the electrophysiological or physiological measuring system, the method may include stop playing the second audio stimulation.

In some embodiments, if the signal indicative of relaxing the facial muscle was not received from the electrophysiological measuring system, repeating steps 230-240 to check if the attention index, and therefore the awareness of the patient is above the threshold value. If the answer is YES, repeat playing the second audio stimulation and the instructions until the patients learns how to relax the facial muscle and stop the second audio stimulation. If the attention index is lower than the threshold value the method may include providing the patient a relaxing stimulation (e.g., low tone monotonic sound, or soft stimulation, etc.) that can cause a relaxation of the facial or eye muscles, thereby associating relaxation of the facial or eye muscles with the command stop the second audio stimulation.

In some embodiments, the method may include providing to user instructions to associate moving of facial or eye muscles with the word YES, and relaxing the facial muscle with the word NO.

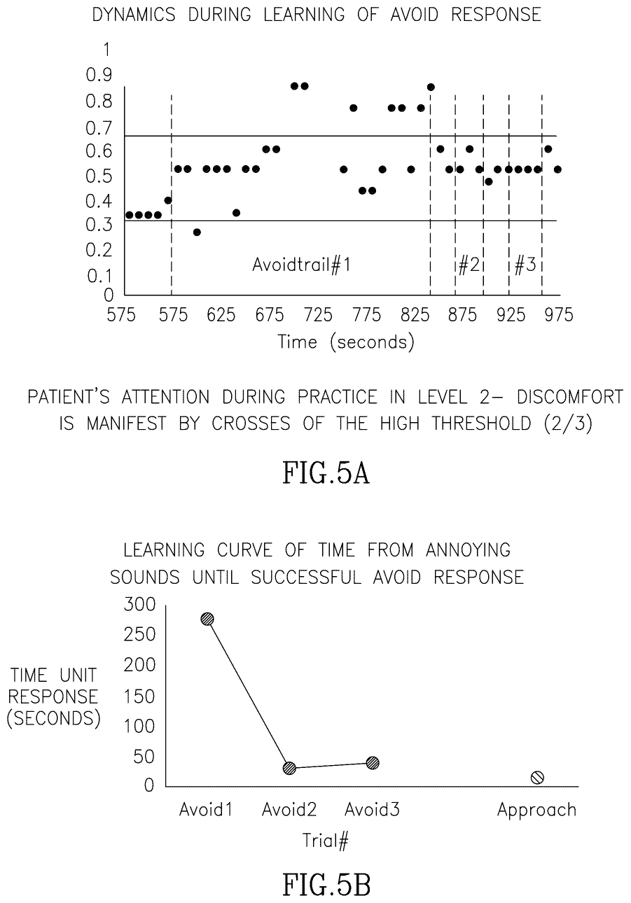

Reference is now made to FIGS. 5A and 5B which are graphs showing the effect of teaching the patient an “avoid” or NO command on the attention indexes of patients with reduced consciousness according to some embodiments of the invention. FIG. 5A shows the attention index during multiple trails of playing to the patient an unpleasant sound. In this specific trial the patient was requested to close his eyes to stop the unpleasant sound. The trails were repeated as long as the attention index is higher than the threshold value. If the attention index is lower than the threshold value an evoking stimulation was used. As shown in the graph in FIG. 5B two trials were required for the patients to be trained to stop the unpleasant sound.

In some embodiments, after teaching a patient diagnosed with DOC to associate the moving of facial or eye muscles with the word YES, and relaxing the facial muscle with the word NO, similar approach may be used to help the patient to communicate using an auditory communication board for augmentative and alternative communication (AAC).

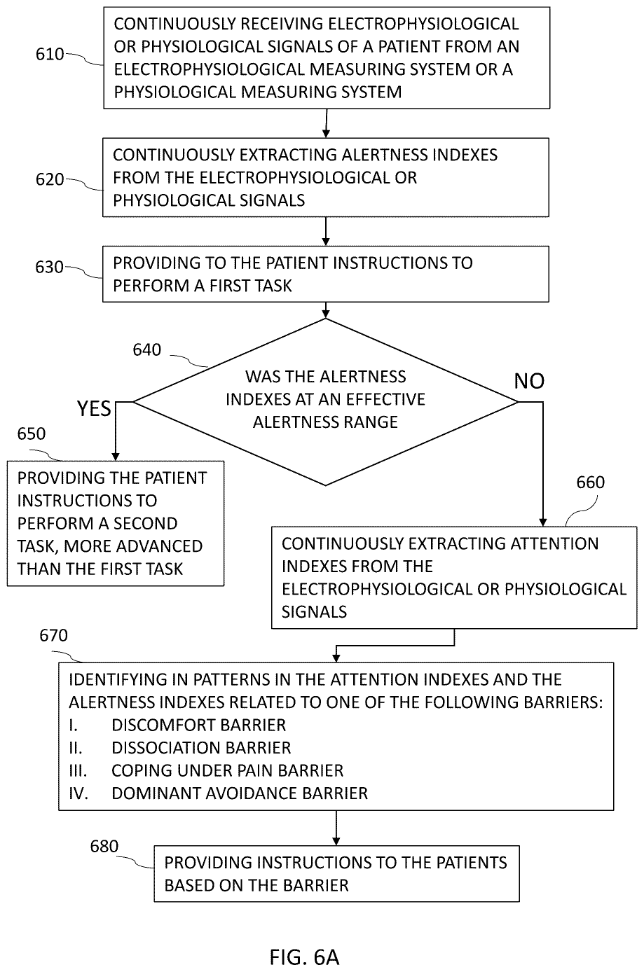

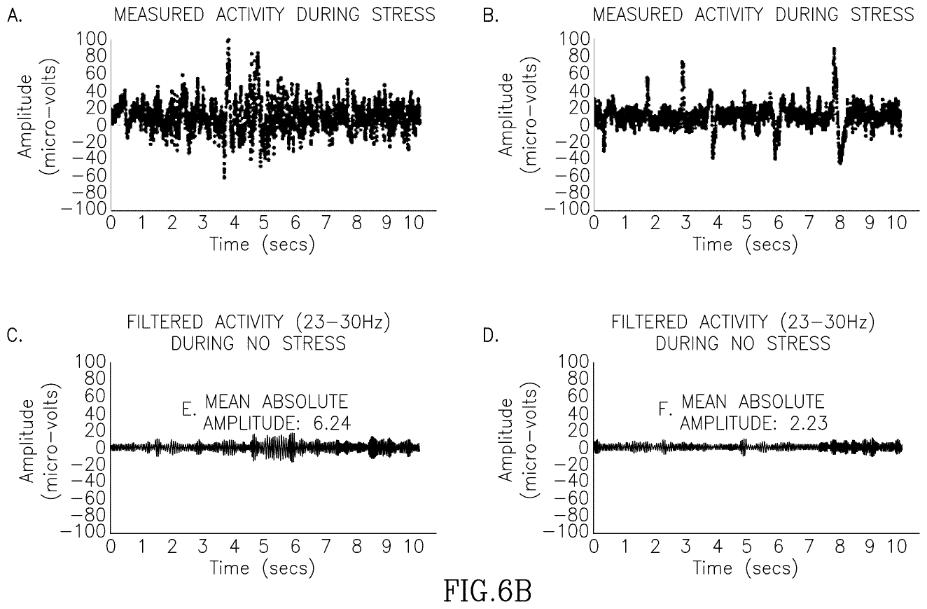

Reference is now made to FIG. 6A which is a flowchart of a method of rehabilitation of at least one of chronic pain, stress, and anxiety according to some embodiments of the invention. The method of FIG. 6A may treat patients with chronic pain (e.g., Complex Regional Pain Syndrome (CRPS), fibromyalgia, myofascial pain, prevalent cases of low back pain, tension headache and migraine, somatoform pain, and the like), chronic anxiety and stress. The method of FIG. 6A may be executed by computing device 10 of system 100.

In step 610, the method may include continuously receiving electrophysiological or physiological signals of a patient from an electrophysiological measuring system or a physiological measuring system selected from, a measuring system, such as system 20, as discussed with respect to step 234 herein above.

In step 620, the method may include continuously extracting alertness indexes from the electrophysiological signals. The alertness indexes may be extracted from the raw signals received from measurements system 20.

Reference is now made to FIG. 6B which shows nonlimiting examples for EEG data taken during tension and relaxed period according to some embodiments of the invention. The graphs of FIG. 6B show: A. raw EEG activity during tension; B. raw EEG activity during relaxion; C. filtered activity during tension; D. filtered activity during relaxion; E. mean activity during tension; and F. mean activity during relaxion. One nonlimiting example for an alertness index may include calculating the ratio between the mean activity during tension and the mean activity during baseline periods without tension for a predetermined period (e.g., 3 minutes) and setting this ratio a reference value for alertness. Thereafter, the tension activity is considered increased for any given period (e.g., any 1 minute), if it is increased by a threshold (e.g., 30%) beyond the reference value. The reference value is dynamic and may change during the test.

There are various methods that may be employed to identify in real-time, at a temporal resolution of a few seconds, an index for the degree of alertness. Some nonlimiting examples may include:
I. Power and power ratio based markers. Increased tension/alertness could be measured from absolute power at the (partial) beta or gamma ranges (one non-limiting example could be seen in FIG. 6B).
II. Template-matching based indexes: Multiple pattern templates were identified as related to increased alertness/tension.
III. Variability based indexes: It has been shown that rapid signal variability among consecutive sample sequences, which last between tens of milliseconds to few seconds, associate with increased alertness/tension.

For all the above types of indexes as well as for other electrophysiological indexes for increased alertness/tension, the data may be analyzed online or offline in segments of a predefined duration, e.g., 2.5 seconds to 10 seconds. Each segment could be divided to epochs of predefined duration, e.g., 0.5 second or 1 second. Prior to the computation of the indexes, signal epochs that were identified as noisy (e.g., due to large-amplitude waves, or by any other means) may be filtered. Therefore, it may be possible to generate indexes only for samples with enough epochs (e.g., more than 50% valid epochs).

In some embodiments, the extraction of alertness indexes may be conducted using any suitable method.

In some embodiments, the indexes may be normalized. A nonlimiting example for normalizing various alertness indexes may include computing for each 10-seconds segment the power of higher beta band activity. Segments in which the CEI value was in the middle third (between ⅓ and ⅔) were considered as baseline segments, and the mean baseline beta power from these segments was updated with every additional baseline segment during the sampling period. In some embodiments, a value TensI for a given segment was derived from the ratio between the power of beta activity in this specific segment and the mean baseline beta power of the baseline segments preceding it. Then the TensI value was divided by two. Accordingly, when the current segment beta power was equal to the mean baseline beta power of the baseline segments preceding it, TensI was 0.5. If TensI was greater than 1 it was set as 1. Thus TensI was limited to the [0,1] range. The normalization to the baseline activity, in segments of middle third CEI, or effective attentional effort follows a pilot experience with multiple clinical patients.

In some embodiments, the alertness indexes and/or the attention indexes discussed herein may all be normalized and thus may have a value ranging between 0 to 1 as shown in FIGS. 4A, 5B, 7, 8A-8C, 10A, 10B and 11B.

In some embodiments, the extraction of alertness indexes may be conducted using signals received from a single channel of system 20.

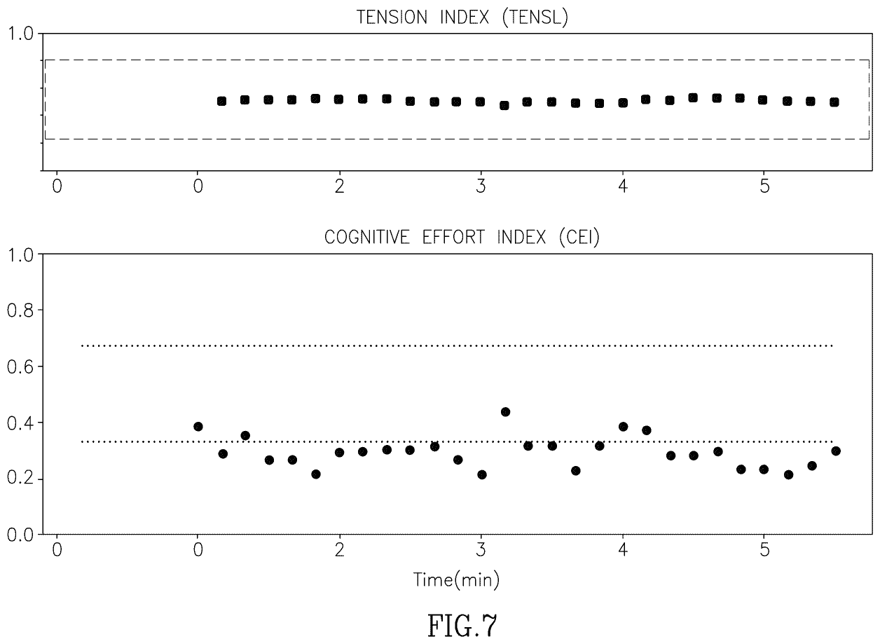

Reference in now made to FIG. 7 that shows a nonlimiting example, for graphs of both alertness index (e.g., tension index) and attention index (e.g., CEI) prior to giving the subject any task. The Patient is relaxed, therefore the alertness index is within alertness index range (marked in dashed lines), for example, between 0.4 and 0.8 and the attention index is above the threshold value of 0.3.

In step 630, the method may include providing to the patient instructions to perform a first task. The task may be a task that may cause the patient pain, anxiety and/or stress. The instructions may be provided as verbal instructions from audio unit 30 or as a written message, an image or animation displayed on display unit 40. The first task may be a first step in performing a larger task. For example, the larger task may be drinking water from a cup standing on a table. Therefore, the first task may be grasping the cup standing on the table. The second task may include also grabbing the cup towards the body of the patient, the third task may include also lifting the cup towards the mouth, and the fourth task may include also drinking water. This exercise may be conducted first with an empty cup, followed by repeating the all tasks with a semi/full cup.

In step 640, controller 2 may determine if the extracted alertness indexes is at an effective alertness range during the performance of the first task (step 640—YES), for example, as shown in FIG. 7.

Therefore, in step 650 the method may include providing the patient instructions to perform a second task, more advanced than the first task. For example, if the patient successfully grasped the cup standing on the table, he may be given the second task to grab the cup towards his/her body.

In some embodiments, if the patient failed in the first attempt, then several repetitions of step 630 may be required for the patient to complete the first task prior to advancing to the second task. The exercise may continue as long as the extracted alertness indexes is at an effective alertness range.

In some embodiments, the alertness indexes may be out of the effective alertness range (Step 640—NO) therefore, the method may include in Step 660, continuously extracting attention indexes from the electrophysiological or physiological signals, for example, as discussed herein above with respect to FIGS. 2, 3A and 3B.

In step 670 the method may include identifying patterns in the attention indexes and the alertness indexes related to one of the following barriers:
I. discomfort barrier, when the attention indexes is below an attention threshold values and the alertness indexes is above the effective alertness range;
II. dissociation barrier, when the attention indexes is below a threshold value and the alertness indexes is below the effective alertness range;
III. coping under pain barrier, when the alertness indexes is above the effective alertness range and the attention indexes is above the threshold value; and
IV. dominant avoidance barrier, when the alertness indexes is at the effective alertness range and the attention indexes is one of: below the threshold value, and above the threshold value while having momentary decreases in the attention indexes to below the threshold value.

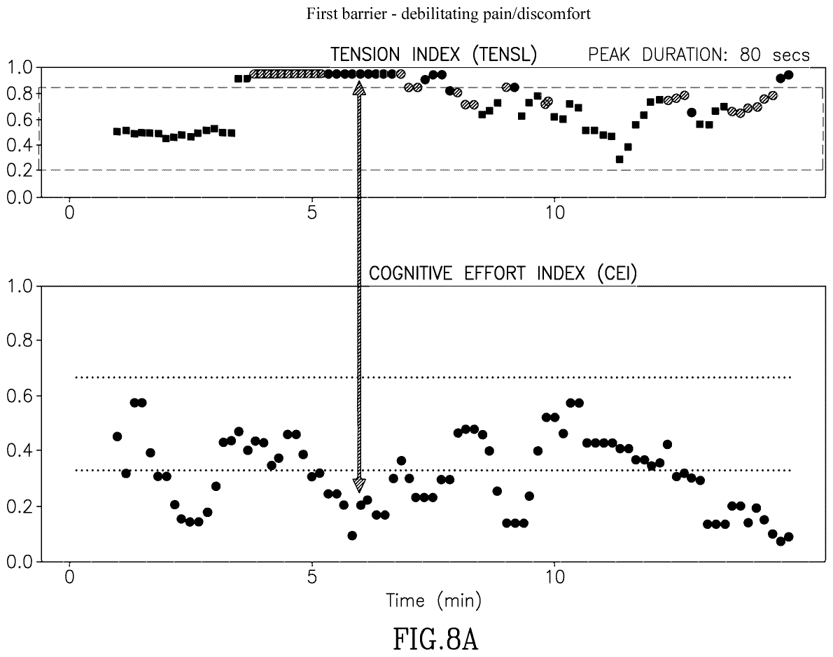

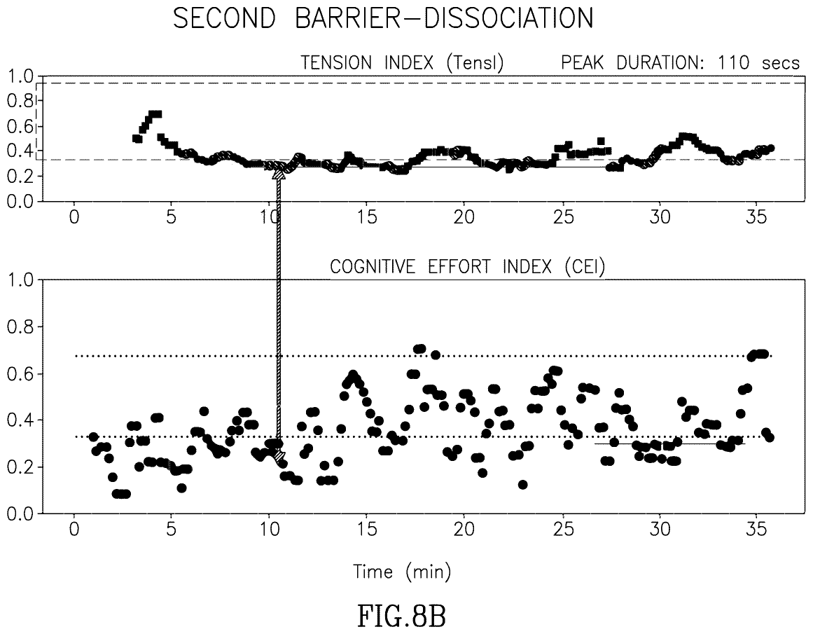

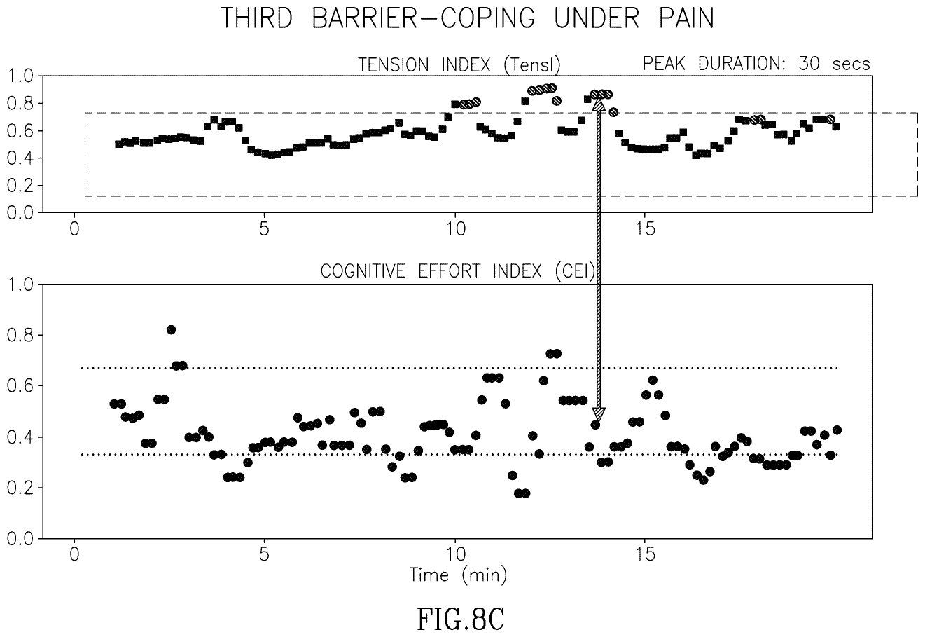

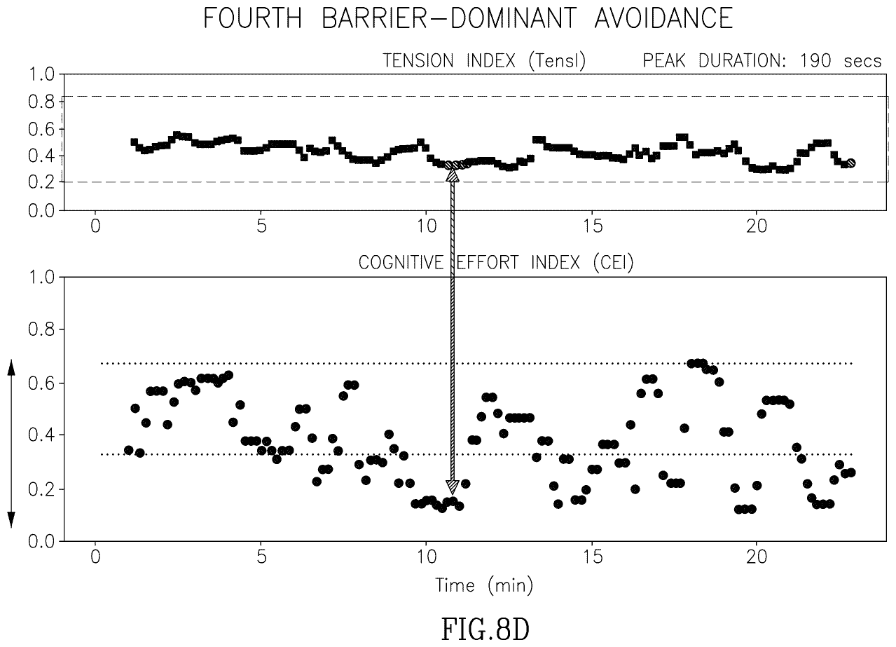

Reference is now made to FIG. 8A which shows attention indexes and alertness indexes of a patient having discomfort (e.g., disabling) barrier during the performance of a task. As shown and marked by the arow the attention indexes is below an attention threshold values (0.3) and the alertness indexes is above the effective alertness range (above 0.8). The discomfort barrier may stop the patient from performing the task. Therefore, the system once identifying a discomfort barrier, may suggest to the patient to do at least one of: (a) rest (e.g., actively relax without continuing with the task) for a first predetermined amount of time; (b) perform a third task, less advanced than the first task; or (c) rest for a second predetermined amount of time and repeat the first task.

Reference is now made to FIG. 8B which includes graphs showing alertness indexes and attention indexes of patients having a dissociation barrier during the performance of a task according to some embodiments of the invention. As shown and marked by the arrow the attention indexes is below a threshold value (below 0.3) and the alertness indexes is below the effective alertness range (below 0.4). The patient is dissociated with the task therefore lacks both the required awareness and alertness to succeed. Therefore, the patient may be asked whether they are in pain/stress/anxiety which causes the dissociation, and then the procedure is as disclosed above with respect to the discomfort Barrier. Alternatively, if they are still OK they will be requested to continue with the task.

Reference is now made to FIG. 8C which includes graphs showing alertness indexes and attention indexes of patients having a coping under pain barrier during e performance of a task according to some embodiments of the invention. As shown and marked by the arow the alertness indexes is above the effective alertness range (above 0.8) and the attention indexes is above the threshold value (above 0.3), meaning that the patient is in full awareness but feels pain/stress while performing the task. Upon determining the coping under pain/stress/anxiety barrier, controller 2 may provide the patient first instructions to actively relax while performing the first task (e.g., take a deep breath while continuing performing the task). If the actively relaxing fails (after one or more attempts) the controller may provide the user one of the following instructions: (a) rest (e.g., actively relax without continuing with the task) for a first predetermined amount of time; (b) perform a third task, less advanced than the first task; or (c) rest for a second predetermined amount of time and repeat the first task.

Reference is now made to FIG. 8D which includes graphs showing alertness indexes and attention indexes of patients during a dominant avoidance barrier according to some embodiments of the invention. As shown and marked by the arrow the alertness indexes is at the effective alertness range (e.g., between 0.4 to 0.8 marked in dashed lines) and the attention indexes is one of: below the threshold value (0.3) or above the threshold value while having momentary decreases in the attention indexes to below the threshold value. In such a case the patient can bear the pain/stress/anxiety but has limited awareness to the action he/she is performing. In such case, his/her attention may need to be evoked by a simulation or instructions, for example, take a deep breath while performing the task. Additionally or alternatively the patients may be asked whether they are in pain/stress/anxiety which causes the dissociation, and then the procedure is as above for the discomfort barrier, or whether they are still OK and wish to continue.

In some embodiments, step 610 to 660 may be repeated until the patient learns to perform the larger task. This may take any time from a couple of minutes to several days depends on the patient's condition. In some embodiments, the selection, duration and repetition of the provided instructions are determined based on the type of barrier.

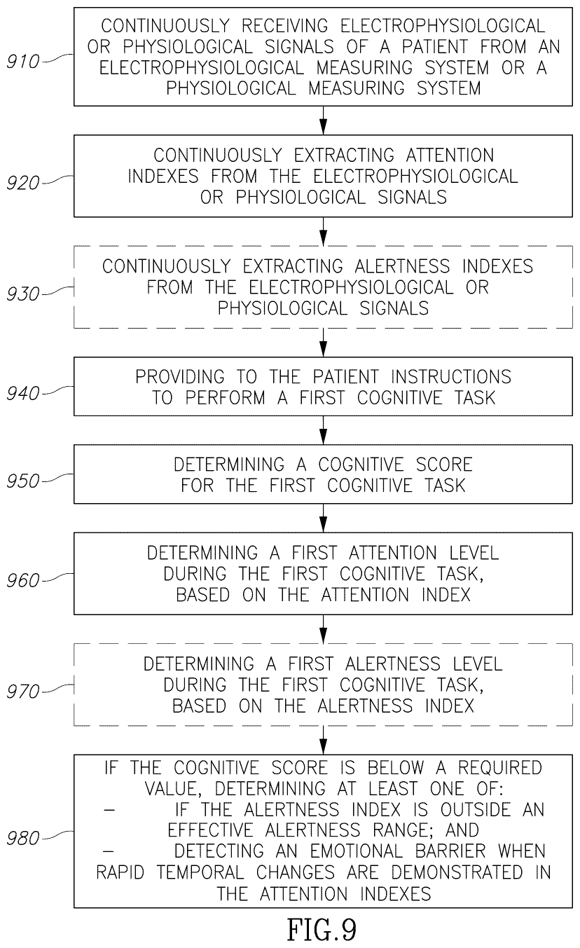

Reference is now made to FIG. 9 which is a flowchart of a method of post-operative rehabilitation according to some embodiments of the invention. The method of FIG. 9 may be used for the rehabilitation of patients with post-operative cognitive dysfunction or decline (POCD). The method of FIG. 9 may be executed by computing device 10 of system 100.

In step 910, the method may include continuously receiving electrophysiological or physiological signals of a patient from an electrophysiological measuring system or a physiological measuring system selected from, a measuring system, such as system 20, as discussed with respect to steps 610 and 234 herein above.

In step 920, the method may include continuously extracting attention indexes from the electrophysiological or physiological signals. Step 920 may be substantially similar to steps 660 and 240 disclosed herein above.

In some embodiments, the method may also include step 930 at which alertness indexes may continuously be extracted from the electrophysiological or physiological signals. Step 930 may be substantially similar to step 620 disclosed herein above.

In step 940, controller 2 may provide the patient instructions to perform a first cognitive task. For example, the controller may display on display unit 40 a computerized version of a Montreal Cognitive Assessment (MOCA) test and instruct (by an audio instruction or textural instruction) the user to take the test.

In step 950, controller 2 may determine a cognitive score for the first cognitive task.

In step 960 controller 2 may determine a first attention level during the first cognitive task, based on the attention index.

In some embodiments, controller 2 may further determine a first alertness level during the first cognitive task, based on the alertness index.

In step 970, if the cognitive score is below a required value detecting an emotional barrier when rapid temporal changes are demonstrated in the attention indexes. In such a case the low score was not achieved due to lack in cognitive abilities, but due to an emotional barrier. Therefore, controller 2 may provide the patents the following instructions (by playing an audio and/or providing visible instructions): (a) relax and breath for a predetermined amount of time if the alertness index is outside the effective alertness range; and (b) repeat the first cognitive task.

Additionally or alternatively, if the cognitive score is below a required value, determining if the alertness index is outside an effective alertness range. If such was not achieved due to lack in cognitive abilities, but due to a stressful state of mind. Therefore, controller 2 may provide the patents the following instructions (by playing an audio and/or providing visible instructions): (a) relax and breath for a predetermined amount of time if the alertness index is outside the effective alertness range; and (b) repeat the first cognitive task.

In some embodiments, controller 2 may be configured to provide the patient stimulation if the attention index is below an attention threshold level. The stimulation may be an audio/visible stimulation.

In some embodiments, controller 2 may provide the patient instructions to perform a second cognitive task if the determined cognitive score is above the required value.

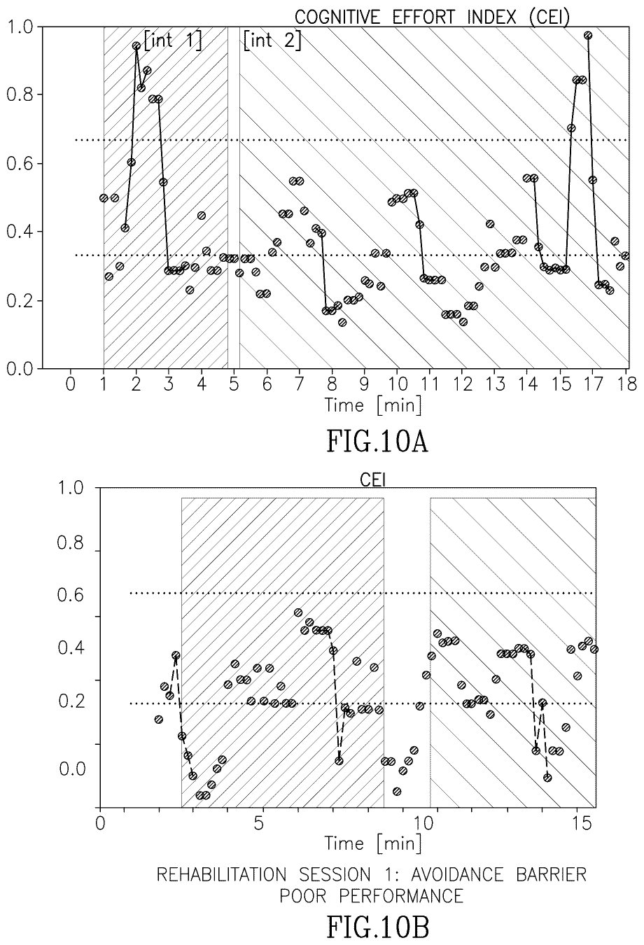

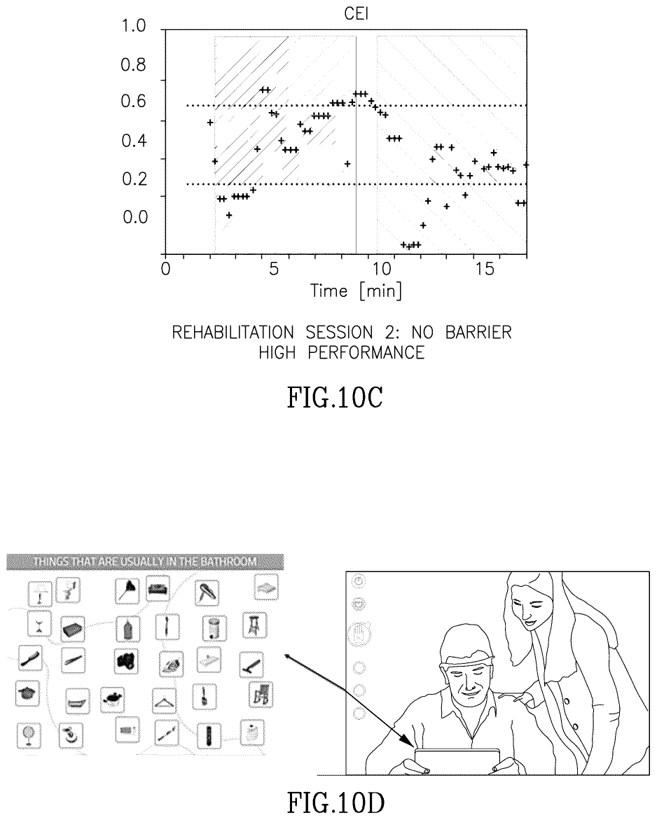

Reference is now made to FIG. 10A which is a graph showing the attention indexes of post-surgery patients according to some embodiments of the invention. In the nonlimiting example of FIG. 10A the patient suffered from a continuous decrease in the cognitive abilities weeks after surgery. It was found that the patient suffers from an emotional barrier (e.g., anxiety) shown by the rapid temporal changes in the attention index. When anxiety was handled the cognitive score has improved.

Reference is now made to FIGS. 10B and 10C include graphs showing post-operative cognitive rehabilitation results according to some embodiments of the invention. FIG. 10B shows the CEI measured during poor cognitive performance due to the avoidance barrier. FIG. 10C shows the CEI of the same patient after 3 rehabilitation sessions showing no avoidance barrier which results in much improve cognitive performance.

Reference is now made to FIG. 10D which includes images of a test board and a patient during post-surgery cognitive exercise according to some embodiments of the invention. These two images are a part of a treatment system, through which the cognitive exercise is presented, and if an emotional barrier is detected (by one of the two ways specified, then recommendations for relaxation are set, and the exercise stops and continues once relaxation and focus are regained.

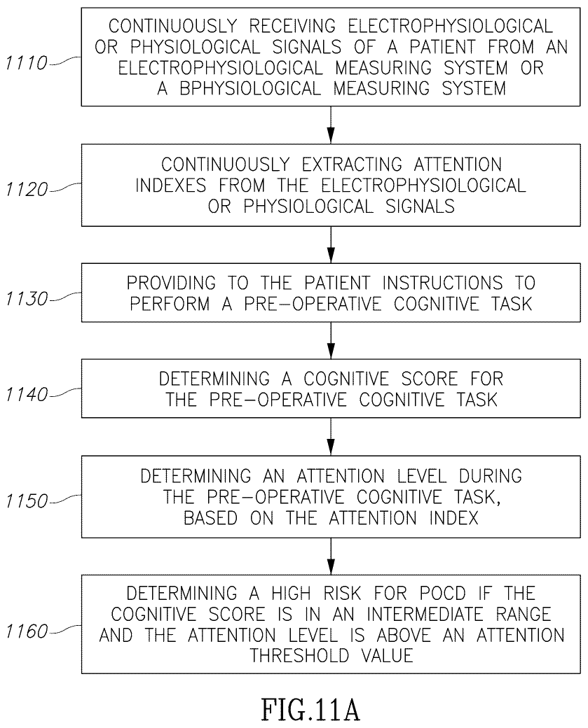

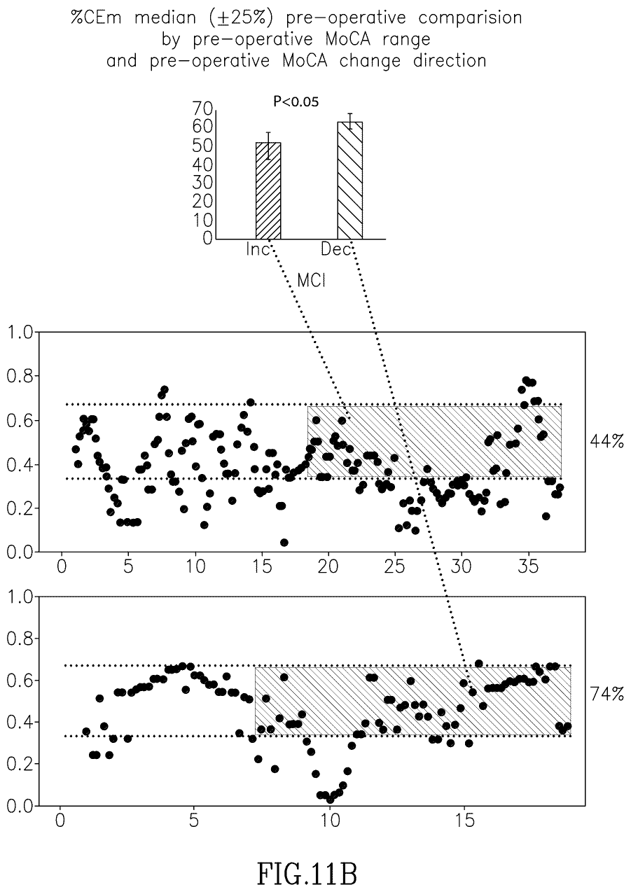

Reference is now made to FIG. 11A which is a flowchart of a method of pre-operative prediction of post-operative cognitive dysfunction or decline (POCD) according to some embodiments of the invention. The method of FIG. 11A may allow to assess if a certain patient is likely to suffer from reduced cognitive abilities following a surgery, prior to the surgery. The method of FIG. 11A may be executed by computing device 10 of system 100.

In step 1110, the method may include continuously receiving electrophysiological or physiological signals of a patient from an electrophysiological measuring system or a physiological measuring system selected from, a measuring system, such as system 20, as discussed with respect to steps 910, 610 and 234 herein above.

In step 1120, the method may include continuously extracting attention indexes from the electrophysiological or physiological signals step 920 may be substantially similar to steps 920, 660 and 240 disclosed herein above.

In step 1130, controller 2 may provide to the patient instructions to perform a pre-operative cognitive task. For example, the controller may display on display unit 40 a computerized version of a Montreal Cognitive Assessment (MOCA) test and instruct (by an audio instruction or textural instruction) the user to take the test.

In step 1140, controller 2 may determine a cognitive score for the pre-operative cognitive task.

In step 1150, controller 2 may determine an attention level during the pre-operative cognitive task, based on the attention index.

In step 1160, controller 2 may determine a high risk for POCD if the cognitive score is in an intermediate range and the attention level is above an attention threshold value. Meaning that in order to gets a readable score, the patient need to put high cognitive effort during to the test. It was found out that these patients have a high risk for POCD, as shown in the graphs of FIG. 11B. In some embodiments, the method may include determining the high risk for POCD if no mental/emotional barrier was detected in the attention indexes, wherein the emotional barrier is defined by rapid temporal changes in the attention indexes.

Reference is now made to FIG. 11B which shows two CEI graphs taken during MOCA test of two patients. The first graph shows a patient having 44% of its CEI points as effective points, while the other has 74% of it's CEI points as effective points. As used herein the effective points are the points in the middle range (between the lower (e.g., 0.35) and upper e.g., (0.7) of the CEI graph. The inventors found that having, for example, 60% or more of effective points may indicate high risk for developing POCD, while having less than 20% effective points may show low risk for developing POCD. Therefore, the second patient with the 74% effective points has a higher risk for developing POCD. As should be appreciated by the one skilled in the art. The thresholds of 20% and 60% are given as an example only, and other thresholds may be suitable as well.

In some embodiments, the high risk for POCD is further based on at least one of: general cognitive background, such as detailed cognitive abilities; pre-operative medical background, such as cardiovascular disease, diabetes, hypertension, smoking, psychiatric morbidities, etc.; And also demographic factors such as age, level of education, etc.

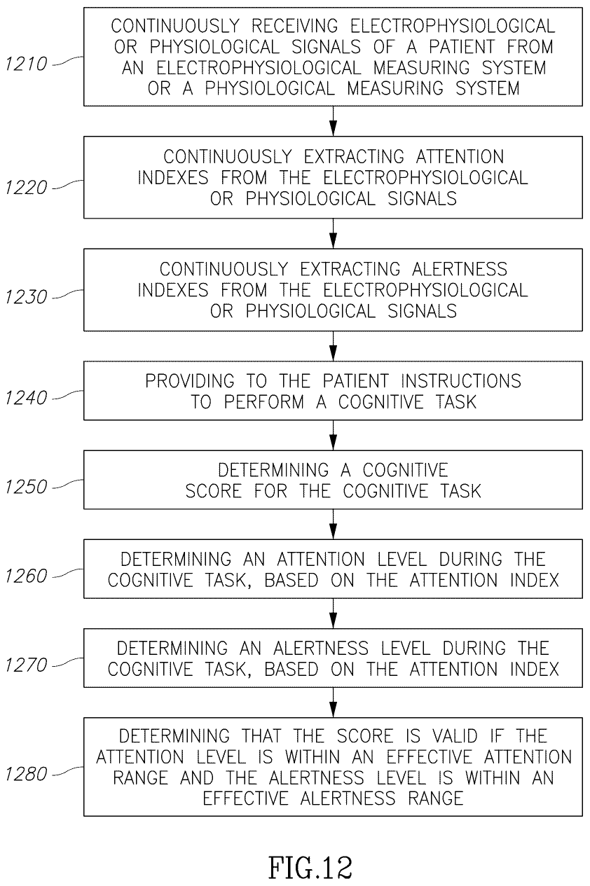

Reference is now made to FIG. 12 which is a flowchart of evaluating the validity of a cognitive test according to some embodiments of the invention. In some embodiments, the score of the test may be affected from the emotional/mental state of the patient (e.g., stress, anxiety, fatigue, etc.). Therefore, patients may be given low cognitive scores that do not present the real cognitive abilities of the patients. The method of FIG. 12 may be executed by computing device 10 of system 100.

In step 1210, the method may include continuously receiving electrophysiological or physiological signals of a patient from an electrophysiological measuring system or a physiological measuring system selected from, a measuring system, such as system 20, as discussed with respect to steps 1110, 910, 610 and 234 herein above.

In step 1220, the method may include continuously extracting attention indexes from the electrophysiological or physiological signals. Step 1220 may be substantially similar to steps 1120, 920, 660 and 240 disclosed herein above.

In step 1230, the method may include continuously extracting alertness indexes from the electrophysiological or physiological signals. Step 1130 may be substantially similar to steps 620 and 930 disclosed herein above.

In step 1240, controller 2 may provide to the patient instructions to perform a cognitive task. For example, the controller may display on display unit 40 a computerized version of a Montreal Cognitive Assessment (MOCA) test and instruct (by an audio instruction or textural instruction) the user to take the test.

In step 1250, controller 2 may determine a cognitive score for the cognitive task.

In step 1260 controller 2 may determine a first attention level during the cognitive task, based on the attention index.

In step 1270, controller 2 may further determine a first alertness level during the cognitive task, based on the alertness index.

In step 1280, controller 2 may determine that the score is valid if the attention level is within an effective attention range and the alertness level is within an effective alertness range. In some embodiments, determining that the score is valid if no emotional barrier was detected in the attention indexes, wherein the emotional barrier is defined by rapid temporal changes in the attention indexes.

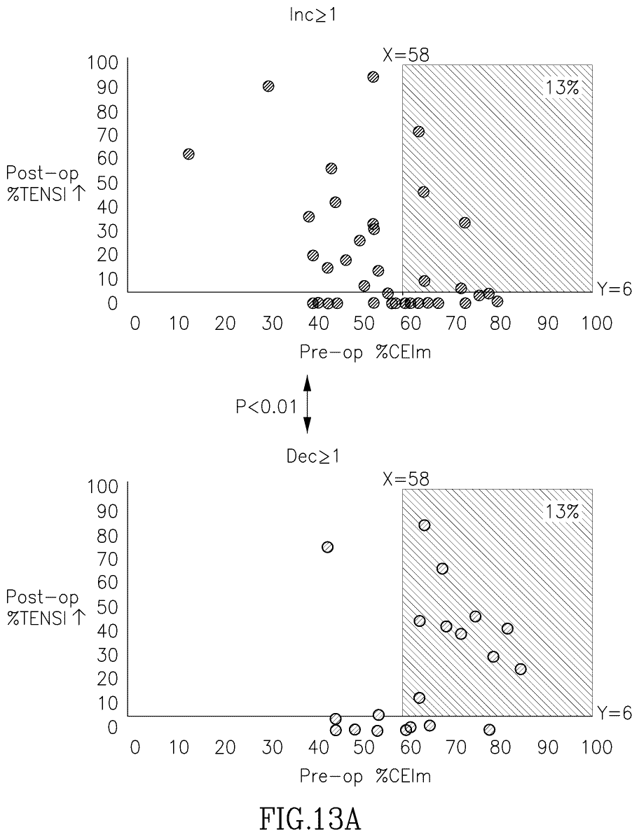

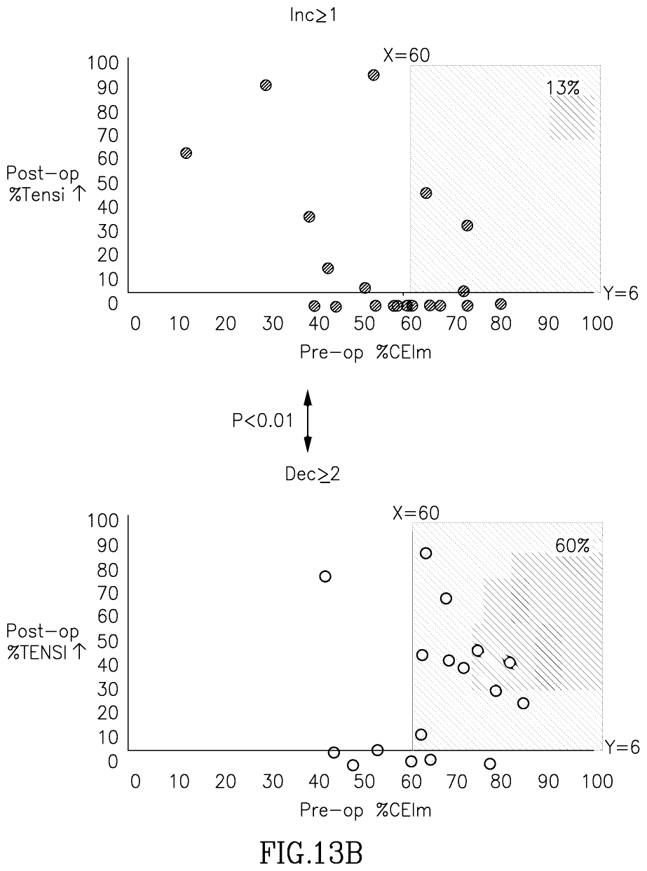

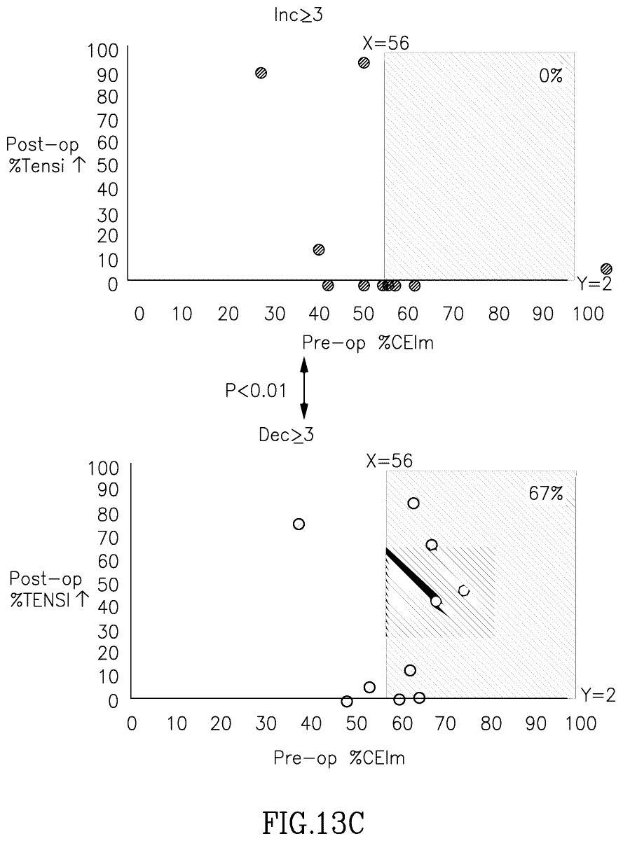

Reference is now made to FIGS. 13A, 13B and 13C which are graphs showing several cognitive scores and attention indexes taken during MOCA tests performed at different levels of attention and alertness according to some embodiments of the invention. The graphs show that patients that improved from the first test to the second test are the ones that invested less effort in the first test and were over-alerted in the second test.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of a Disorder of Consciousness (DOC) rehabilitation comprising:
a. providing a patient, a first audio stimulation during a first predetermined duration;
b. at the end of the first audio stimulation, providing the patient instruction to move facial or eye muscles in order to replay the audio stimulation; and
c. replaying the first audio stimulation if a signal indicative of a voluntary response of the facial or eye muscles is received.

2. The method of claim 1, further comprising
a. continuously receiving electrophysiological or physiological signals of a patient from an electrophysiological measuring system or a physiological measuring system selected from, an Electroencephalogram (EEG) system, an Electromyography (EMG) system, electrooculogram (EOG); or an eye-tracking system,
b. continuously extracting attention indexes from the electrophysiological or physiological signals;
c. detecting a first attention index higher than a threshold value;
d. if the signal indicative of the voluntary responses was not received, providing the patient additional instruction to move facial or eye muscles in order to replay the audio stimulation;
e. replaying the first audio stimulation if a signal indicative of a voluntary response of the facial or eye muscles is received.

3. The method of claim 1, further comprising:
a. continuously receiving electrophysiological or physiological signals of a patient from an electrophysiological measuring system or a physiological measuring system selected from, an Electroencephalogram (EEG) system, an Electromyography (EMG) system, electrooculogram (EOG); or an eye-tracking system,
b. continuously extracting attention indexes from the electrophysiological or physiological signals;
c. detecting a second attention index lower than the threshold value;
d. if the signal indicative of the voluntary responses was not received, providing to the patient an evoking stimulation configured to evoke a response of the facial or eye muscles;
e. replaying the first audio stimulation.

4. The method of claim 1, wherein the signal indicative of the voluntary response is received from the electrophysiological or physiological measuring system.

5. The method of claim 4, wherein the signal indicative of the voluntary response is received from the same channel as the electrophysiological or physiological signals.

6. The method of claim 1, wherein the instruction to move the facial or eye muscles is provided as one of: audio instruction, visual instruction, and muscle stimulation.

7. The method of claim 6, wherein the muscle stimulation is provided by an electrophysiological measuring system or a physiological measuring system.

8. The method of claim 1, wherein the voluntary responses of the facial or eye muscles comprising at least one of: blinking, closing the eyes, raising the eyebrows, directing gaze, smiling, opening and closing the mouth.

9. The method of claim 1, further comprising:
a. providing the patient a second audio stimulation during a second predetermined duration;
b. during the second audio stimulation, providing the patient instruction to relax the facial muscle in order to stop the second audio stimulation;
c. stop playing the second audio stimulation if a signal indicative of relaxing the facial muscle is received from the electrophysiological or physiological measuring system.

10. The method of claim 9, wherein the second audio stimulation includes an unpleasant sound.

11. The method of claim 9, wherein the instruction to relax the facial muscle is provided as one of: audio instruction, visual instruction, and muscle stimulation.

12. The method of claim 9, further comprising:
providing to user instructions to associate the moving of facial or eye muscles with the word YES, and relaxing the facial muscle with the word NO.

13. The method of claim 1 wherein the extraction of an attention index comprises dividing the electrophysiological signal into a plurality of segments and dividing each of the segments into a plurality of epochs.

14. The method of claim 13 wherein the duration of each of the plurality of the segments is in the range of 2.5 to 10 seconds and the duration of each of the epochs is in the range 0.2 to 1 seconds.

15.-33. (canceled)

34. A system for a Disorder of Consciousness (DOC) rehabilitation comprising:
an audio unit;
an electrophysiological measuring system or a physiological measuring system for measuring electrophysiological or physiological signals of a patient; and
a controller configured to:
a. control the audio unit to provide to a patient a first audio stimulation during a first predetermined duration;
b. at the end of the first audio stimulation, control the audio unit to provide to the patient instruction to move facial or eye muscles in order to replay the audio stimulation; and
c. control the audio unit to replay the first audio stimulation if a signal indicative of a voluntary response of the facial or eye muscles is received from the an electrophysiological measuring system or the physiological measuring system.

35. The system of claim 34, wherein the controller is further configured to:
a. continuously receive electrophysiological or physiological signals of a patient from the electrophysiological measuring system or the physiological measuring system selected from, an Electroencephalogram (EEG) system, an Electromyography (EMG) system, electrooculogram (EOG); or an eye-tracking system,
b. continuously extract attention indexes from the electrophysiological or physiological signals;
c. detect a first attention index higher than a threshold value;
d. if the signal indicative of the voluntary responses was not received, control the audio unit to provide to the patient additional instruction to move facial or eye muscles in order to replay the audio stimulation;
e. control the audio unit to replay the first audio stimulation if a signal indicative of a voluntary response of the facial or eye muscles is received.

36. The system of claim 34, wherein the controller is further configured to:
a. continuously receive electrophysiological or physiological signals of a patient from the electrophysiological measuring system or the physiological measuring system selected from, an Electroencephalogram (EEG) system, an Electromyography (EMG) system, electrooculogram (EOG); or an eye-tracking system,
b. continuously extract attention indexes from the electrophysiological or physiological signals;
c. detect a second attention index lower than the threshold value;
d. if the signal indicative of the voluntary responses was not received, control the audio unit to provide to the patient an evoking stimulation configured to evoke a response of the facial or eye muscles;
e. control the audio unit to reply the first audio stimulation.

37. The system of claim 34, wherein the signal indicative of the voluntary response is received from the electrophysiological or physiological measuring system.

38. The system of claim 34, wherein the controller is further configured to:
a. control the audio unit to provide to the patient a second audio stimulation during a second predetermined duration;
b. during the second audio stimulation, control the audio unit to provide to the patient instruction to relax the facial muscle in order to stop the second audio stimulation;
c. control the audio unit to stop playing the second audio stimulation if a signal indicative of relaxing the facial muscle is received from the electrophysiological or physiological measuring system.
